(19) United States
(12) Patent Application Publication
(10) Pub. No.: US 2013/0144223 A1
(43) Pub. Date: Jun. 6, 2013

(54) NEURAL DRUG DELIVERY SYSTEM WITH MICROVALVES

(71) Applicants: Nicholas D. Hewitt, Chelsea, MI (US); Jamille Hetke, Brooklyn, MI (US); Daryl R. Kipke, Dexter, MI (US); Rio J. Vetter, Ypsilanti, MI (US); John Seymour, Ann Arbor, MI (US)

(72) Inventors: Nicholas D. Hewitt, Chelsea, MI (US); Jamille Hetke, Brooklyn, MI (US); Daryl R. Kipke, Dexter, MI (US); Rio J. Vetter, Ypsilanti, MI (US); John Seymour, Ann Arbor, MI (US)

(21) Appl. No.: 13/556,689

(22) Filed: Jul. 24, 2012

Related U.S. Application Data
(60) Provisional application No. 61/511,353, filed on Jul. 25, 2011.

(51) Int. Cl. A61M 25/00

(52) U.S. Cl. CPC A61M 25/0021; A61M 25/0009; A61M 25/0012; A61M 25/0015
USPC 604/246; 29/890.126

(57) ABSTRACT

An apparatus comprises a tubular body having a lumen and a distal region, a plurality of ports at the distal region of the tubular body, and a plurality of independently gatable microvalves disposed at the plurality of ports. A port extends from internal to the lumen to outside the tubular body, and a gatable microvalve is controllable by a stimulus to provide and prevent fluidic transfer through the ports.

### CLAIM OF PRIORITY

This application claims the benefit of priority under 35 U.S.C. §119(e) of Hewitt et al., U.S. Provisional Patent Application Ser. No. 61/511,353, filed Jul. 25, 2011, which is incorporated by reference herein in its entirety.

### TECHNICAL FIELD

This invention relates generally to the medical field, and more specifically to an improved neural drug delivery system in the medical field.

### BACKGROUND

For many complex neural disease conditions, such as epilepsy and malignant brain tumors, there is a growing technical and clinical rationale to develop therapeutic treatments involving highly controllable, targeted drug delivery. In this approach, the objective is to deliver a therapeutic agent to the central nervous system with precise spatial or regional selection, and to precisely deliver the therapeutic agent at appropriate dosage levels over the appropriate amount of time. However, current conventional drug delivery devices are unable to overcome the complexities of targeted drug delivery. Thus, there is a need in the medical field for improved neural drug delivery.

### BRIEF DESCRIPTION OF THE FIGURES

FIG. 1 shows portions of an example of a device to provide targeted drug delivery, consistent with some example embodiments of the invention.

FIG. 2 shows an example of portions of a system to provide targeted drug delivery, consistent with some example embodiments of the invention.

FIGS. 3-8 show examples of a gatable microvalve, consistent with some example embodiments of the invention.

FIG. 9 shows portions of examples of movable valve flaps, consistent with some example embodiments of the invention.

FIGS. 10-11 show additional examples of gatable microvalves, consistent with some example embodiments of the invention.

FIG. 12 shows an example of a thin film solenoid, consistent with some example embodiments of the invention.

FIG. 13 shows portions of still another example of a gatable microvalve, consistent with some example embodiments of the invention.

FIG. 14 is a flow diagram of a method of making a device to provide targeted drug delivery.

FIG. 15 shows an example of forming portions of a device to provide targeted drug delivery.

FIG. 16 shows another example of forming portions of a device to provide targeted drug delivery.

### DETAILED DESCRIPTION

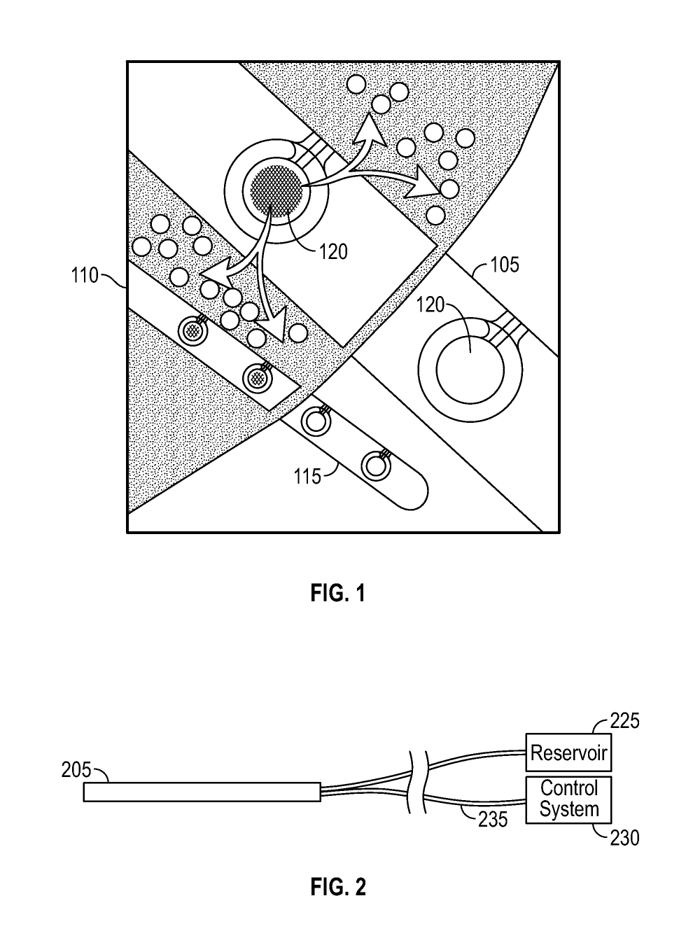

FIG. 1 shows an example of a device 105 to provide targeted drug delivery. The device 105 includes a tubular body 110 having a lumen and a distal region 115. The tubular body 110 includes a plurality of ports at its distal region 115. A port extends from internal to the lumen to the outside of the tubular body to allow release of a fluid contained inside the lumen. In some examples, the device 105 is a microcatheter and the fluid to be released contains a drug. The device 105 also includes a plurality of independently gatable microvalves 120 disposed at the plurality of ports. A gatable microvalve 120 is controllable, or gated, by a stimulus in order to provide and to prevent fluidic transfer through a port. The stimulus can be an electrical stimulus (e.g., an electrical signal) or the stimulus can be a temperature stimulus (e.g., a temperature changing fluid). The dark port represents an open microvalve and the lighter port represents a closed microvalve. A port can have any shape such as circular, square, or rectangular for example. The gatable valve is referred to as a microvalve because of the small size. For instance, a port may have a circular shape and have a diameter of about 100-200 micrometers (μm).

FIG. 2 shows an example of portions of a system 200 to provide targeted drug delivery. The system 200 includes a microcatheter 205 and a reservoir 225. The reservoir 225 supplies a fluidic therapeutic agent to a lumen contained in a tubular body of the microcatheter 205. The microcatheter 205 can be implantable into neural tissue and can include a plurality of ports through which the therapeutic agent may be selectively released to the neural tissue. The microcatheter 205 includes a plurality of microvalves that are individually controllable to provide fluid transfer to a tissue target. The system 200 also includes a control system or control subsystem 230 to provide independent control of the microvalves. The microvalves are gatable in that they can be individually controlled to open and close to provide fluidic transfer and prevent fluidic transfer.

In some examples, the microvalves are electrically controllable using the control subsystem 230. The microcatheter 205 may include electrical conductors (e.g., electrically conducting traces) electrically coupled to the microvalves and extending to the proximal end of the tubular body of the microcatheter 205. The electrical conductors may extend to the control subsystem 230 or the electrical conductors can be bonded to interconnect 235 (e.g., one or more leads) that are electrically coupled to the control subsystem 230. These interconnects can carry electrical signals between the control subsystem 230 and the microvalves. The control subsystem 230 may include one or more of hardware, software, and firmware to perform the functions described. The control subsystem 230 may include logic circuits or a processor (e.g., a microprocessor) to provide an electrical signal or signals to cause actuation of a microvalve.

The neural drug delivery system 200 may be one or both of programmable and manually-controlled by a user to selectively release fluid from the microcatheter to one or more localized regions or the neural drug delivery system may be chemo-responsive to its environment, or may be controlled in any suitable manner. The microcatheter 205 is preferably insertable into neural tissue such as the brain, and provides selective and adjustable pressure-driven drug infusion from discrete locations on the microcatheter 205, such as for therapeutic treatment of epilepsy or brain tumors. The drug infusion may additionally and/or alternatively be driven by any suitable mechanism, and the microcatheter 205 may alternatively be insertable into any suitable tissue for any suitable application. The neural drug delivery system 200 provides consistent, predictable and controllable flow rates of the therapeutic agent, for acute and/or chronic use applications. Furthermore, the neural drug delivery system 200 includes microvalve actuation that is responsive, reacting quickly to changes such as in drug concentration, dosage needs and patient condition.

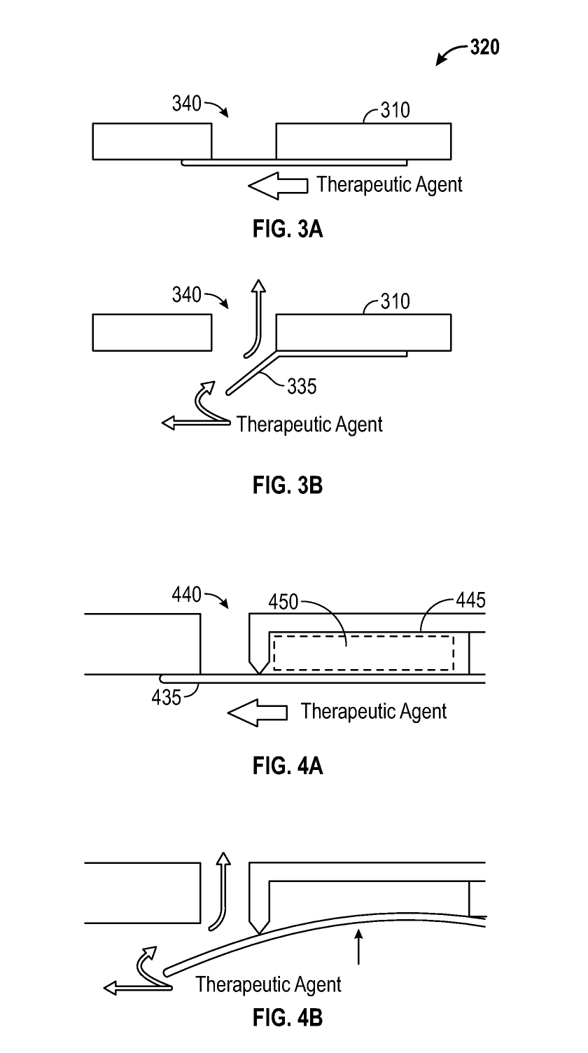

FIGS. 3A and 3B show an example of a gatable microvalve. The microvalve includes a movable valve flap 335 that covers a corresponding port 340 in a wall of the tubular body 310 that forms the lumen. The movable flap is of similar dimension as a port (e.g., 100-200 μm). The movable flap 335 is independently and reversibly deflectable from a closed mode that prevents release of the therapeutic agent from the microcatheter to the tissue to an open mode that provides release of the therapeutic agent from the microcatheter to the tissue along a gradient of therapeutic agent flow rate. As shown in the Figures, the movable valve flap 335 may cover the port 340 on a side of the port 340 internal to the lumen. In other variations, the movable valve flap 335 may cover the port 340 on a side of the port 340 external to the tubular body 340.

The microvalve 320 may include a valve actuator that selectively deflects the flap valve from the closed mode to the open mode. For instance, the movable valve flap 335 may include a polymer material configured to actuate according to an electrical signal, such as by an electrical signal from the control subsystem 230 of FIG. 2.

The microcatheter 205 of FIG. 2 may also include an array of electrode sites in the region of the ports. The electrode sites may be suitable for one or more of recording sensed signals, stimulation of neural target tissue, and making impedance measurements. The electrode sites may be electrically coupled to electrical conductors to provide electrical communication with control subsystem 230. Sensing signals using the electrode sites may aid placement of the microcatheter in the tissue. Sensing of one or both of neural signals and impedance using the electrode sites may enable feedback control of delivery of the therapeutic agent to the tissue. The neural therapy may include a combination of the electrical stimulation with the electrode sites and the therapeutic agent.

The microcatheter 205 can be implantable in tissue and functions to transport a fluid, such as a therapeutic agent, toward targeted regions within the tissue. The microcatheter 205 may be coupled to the fluid reservoir 225, a controllable infusion pump, or other device inside and/or outside the body that provides the therapeutic agent to the microcatheter 205. The microcatheter 205 can be a tubular body having a thin wall and narrow diameter, which may allow the neural device to be minimally invasive and reduce tissue damage during implantation. The microcatheter 205 can be made of a flexible material, but may alternatively be made of a rigid or semi-rigid material. The microcatheter 205 includes a lumen configured to carry the fluidic therapeutic agent, and defines a plurality of ports through which the therapeutic agent may be selectively released to the neural tissue. The lumen (or another second lumen defined by the microcatheter 205) may be used to carry a stylet that aids in positioning the microcatheter 205 during implantation in tissue. The ports provide fluidic communication between the lumen and outside the microcatheter 205. The ports may be arranged longitudinally along, and/or circumferentially around the microcatheter 205. In some embodiments, the distal end of the microcatheter 205 may additionally and/or alternatively include a port. Although the example shown in FIG. 2 shows that the neural drug delivery system includes one microcatheter, in some variations the neural drug delivery system 200 may include multiple separately or jointly controllable microcatheters, such as for simultaneously treating multiple target regions of tissue.

The plurality of gatable microvalves function to selectively allow transfer of fluid through the ports, from inside the microcatheter 205 to the tissue. Each microvalve can be coupled to a respective port such that each of the microvalves is independently and reversibly gatable from a closed mode that prevents release of the fluid from the microcatheter 205 to the tissue to an open mode that allows release of the fluid from the microcatheter 205 to the tissue. The release of the fluid can be controllable along a gradient of therapeutic agent flow rate approximately corresponding to the degree to which the microvalve is open. The microvalve can be biased in the closed mode (e.g., shape biased or biased by an applied voltage), but may alternatively be biased in the open mode or unbiased in either mode. In a preferred embodiment, the neural device includes a one-to-one (1:1) correspondence between microvalves and ports. However, in an alternative variation the neural drug delivery system 200 may include more ports than microvalves (e.g. some ports are not coupled to a microvalve and freely release fluid, or some microvalves are coupled to multiple ports). In another alternative variation, the neural device may include more microvalves than ports (e.g. more than one flap-type microvalve is coupled to a port, such as for redundancy).

As explained previously, a microvalve can include a movable valve flap. The valve flaps of the microvalves can be identically positioned relative to their respective ports, or a portion of the valve flaps may be of one position variation while another portion of the flap valves may be of another position variation. In the example of FIGS. 3A and 3B, the deflectable flap “swings” or folds around approximately a single axis to transition between the closed and open modes.

FIGS. 4A and 4B show another variation of a gatable microvalve. The microvalve again includes a movable valve flap 435. The valve flap 435 can be deflectable and can define a valve actuator cavity 445 between the flap and the wall of the microcatheter, and the flap extends past a fulcrum point of the valve actuator cavity 445, such that the valve actuator cavity 445 is deformable by deflection of the flap around the fulcrum point. As shown in FIG. 4A, in the closed mode the flap is undeflected and is “balanced” on the fulcrum point. As shown in FIG. 4B, in the open mode the deflected flap pivots on the fulcrum point (simultaneously contracting or reducing the size of the valve actuator cavity 445) to create an opening for fluidic access through the port, thereby enabling fluidic access to the port of the microcatheter. In both of these and other variations, the open mode allows fluid to pass out of the microcatheter at a flow rate that approximately corresponds to the degree of flap folding or deflection, which affects the size of the opening that is created.

The example microvalves shown in the Figures can include a valve actuator 450. A valve actuator of a microvalve can function to selectively actuate the flap of the microvalve from the closed mode to the open mode. In other variations, a valve actuator may additionally and/or alternatively actuate the microvalve from the open mode to the closed mode. For example, in an alternative variation a second valve flap may cooperate with the first valve flap, such that the second valve flap has an opposite direction of actuation as the first valve flap, thereby functioning as a locking mechanism for the first valve flap. Valve actuators can be independently operable, but may alternatively be operatively grouped such that one signal activates more than one valve actuator. In the example shown in FIG. 4A, the valve actuator 450 may include specific layered materials preferably located in the valve actuator cavity 445 defined by the movable flap 435 and the wall of the microcatheter, but the valve actuator 450 may alternatively be located in any suitable location solely on the flap, other portion of the flap valve, the microcatheter wall, within the port, or another suitable structure. The valve actuator 450 can include a thin film layering of materials that can be formed during the manufacturing process of the microcatheter, but alternatively the thin film layering may be formed in any suitable process. The thin film layered materials in the valve actuator cavity 445 can induce strains in the flap in response to a particular stimulus. The valve actuator 450 may include one or more of several variations of mechanisms.

In some variations, the valve actuator of the microvalve includes a shape memory alloy material that transitions between martensitic and austenitic phases. Thermomechanical cycling of the shape memory allow material can “train” the material to respond with a given strain in response to a stimulus (such as a temperature change or electrical current), allowing the material to transition between martensitic and austenitic phases without external stress. The shape memory alloy may have “one-way” shape memory (with one original memory shape) or “two-way” shape memory (with two original memory shapes each corresponding to a particular environment or stimulus). For example, the original memory shape of a “one-way” shape memory material may correspond to a default closed mode or to a default open mode of the valve flap. As another example, one original memory shape of a “two way” shape memory material may correspond to the closed mode of the flap valve and the other original memory shape may correspond to the open mode of the flap valve. The valve actuator 450 can include nitinol as the shape memory alloy, but may alternatively include any suitable shape memory alloy. The nitinol may be spray-coated with Teflon or other suitable coating, such as to prevent release of incidental molecules such as nickel ions. In certain examples, the valve actuator 450 can be located on the valve flap 435 outside of a valve actuator cavity 445.

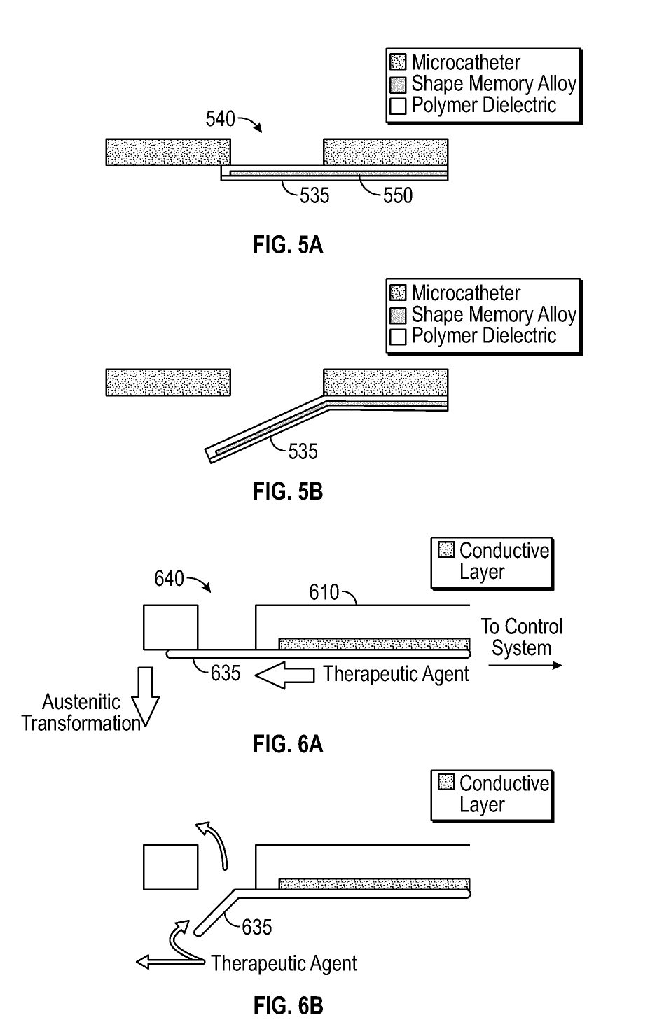

FIGS. 5A and 5B show another variation of a gatable microvalve having a movable valve flap 535. The valve actuator 550 includes a layer of shape memory alloy material coupled to or embedded within the valve flap 535, such that when an electrical stimulus (e.g., a current) or a temperature stimulus (e.g., a temperature change) is applied, the shape memory alloy folds or swings to create an opening to the port 540.

FIGS. 6A and 6B show still another variation of a gatable microvalve. The microvalve includes a movable valve flap 635 and the valve actuator includes a layer of shape memory alloy material coupled to or embedded within the valve flap 635. The shape memory alloy material has a rigid original memory shape as a bent flap in the open mode. A current or other electrical signal can be applied to an electrical conductor near or adjoining the valve flap 635. When a current or other electrical signal (e.g., applied by a control subsystem) raises the temperature of the shape memory alloy flap above body temperature (e.g. 50-60° C.), the valve flap undergoes austenitic transformation having its bent original memory shape, thereby transitioning from the closed mode to the open mode to create an opening to the port 640 in the tubular body 610. The valve flap may open quickly and close relatively slowly, and may rely on pressure gradient between the greater pressure internal to the microcatheter and lesser pressure external to the microcatheter to enforce or hasten transition to the closed mode, such as to bias the microvalve in the closed mode.

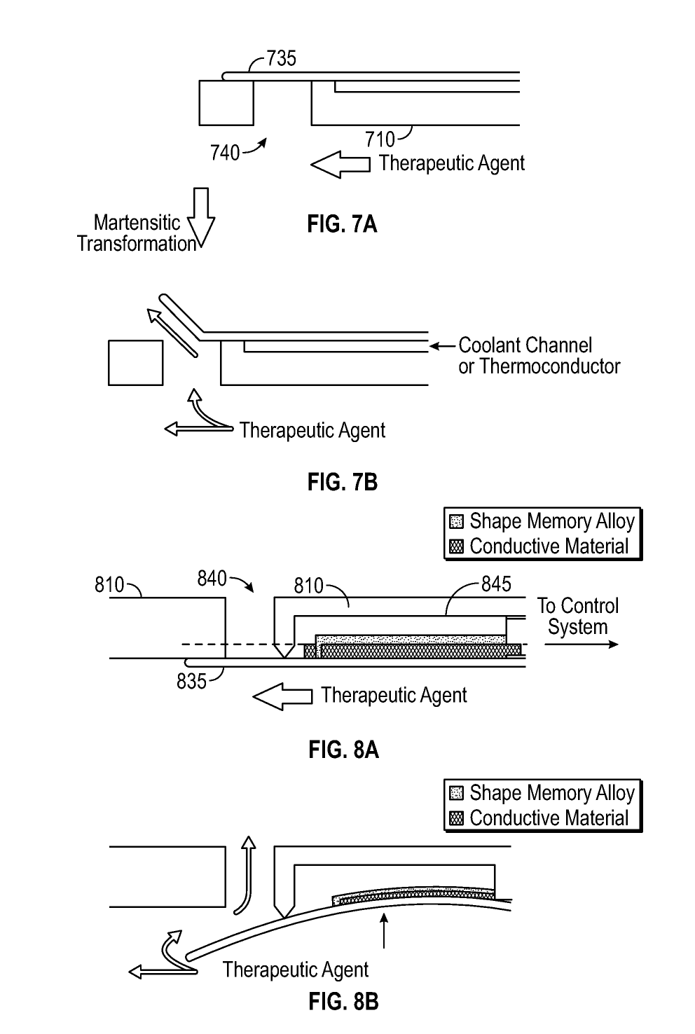

FIGS. 7A and 7B show still another variation of a gatable microvalve. As in FIGS. 6A and 6B, the valve actuator includes a layer of shape memory alloy material coupled to or embedded within the valve flap 735. The shape memory alloy material has a rigid original memory shape as a bent flap as a closed flap in the closed mode. When the valve flap 735 is cooled (e.g., a temperature stimulus such as with coolant or a thermoconductive material) below body temperature (e.g. 5-20° C.), the valve flap 735 undergoes martensitic transformation to become flexible, thereby transitioning from the closed mode into the open mode to create an opening to the port 740 of the tubular body 710.

FIGS. 8A and 8B show still another variation of a gatable microvalve. The microvalve has a valve actuator that includes a layer of a shape memory alloy material coupled to the valve flap 835 within a valve actuator cavity 845, such that when a current or temperature change is applied, the shape memory alloy material expands and deflects the valve flap 835 inwards about a fulcrum to create an opening to the port 840. In other words, the volumetric change in the shape memory alloy material bends the surface of the valve flap 835 on one side of the fulcrum and causes an opposite flexion in the flap on an opposite side of the fulcrum; this resultant flexion creates the opening to the port and allows fluid to pass through the port 840 of the tubular body 810.

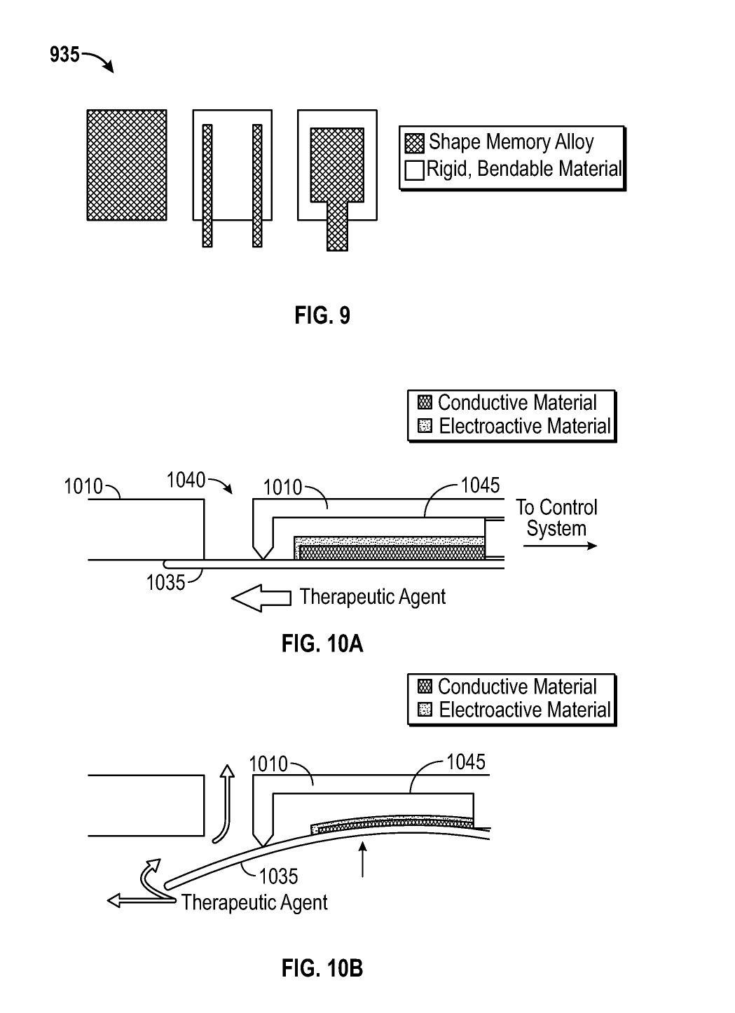

In any of these versions of a valve actuator shown in the Figures, the microvalve structure, and particularly the flap of the microvalve, can be formed from thin-film dielectrics and a thin-film shape memory alloy. Alternatively, as shown in FIG. 9, the valve flap 935 may include wires of the shape memory alloy coupled directly to the flap to actuate the valve flap 935, and/or include an entire sheet of shape memory alloy to form at least a substantial portion of the valve flap 935. In this example, the microvalve independently may open slowly and close relatively quickly, and may rely on shape memory to bias the microvalve in the closed mode. Furthermore, in this example the microvalve may rely on pressure gradient between the greater pressure internal to the microcatheter and lesser pressure external to the microcatheter to enforce or hasten transition to the open mode.

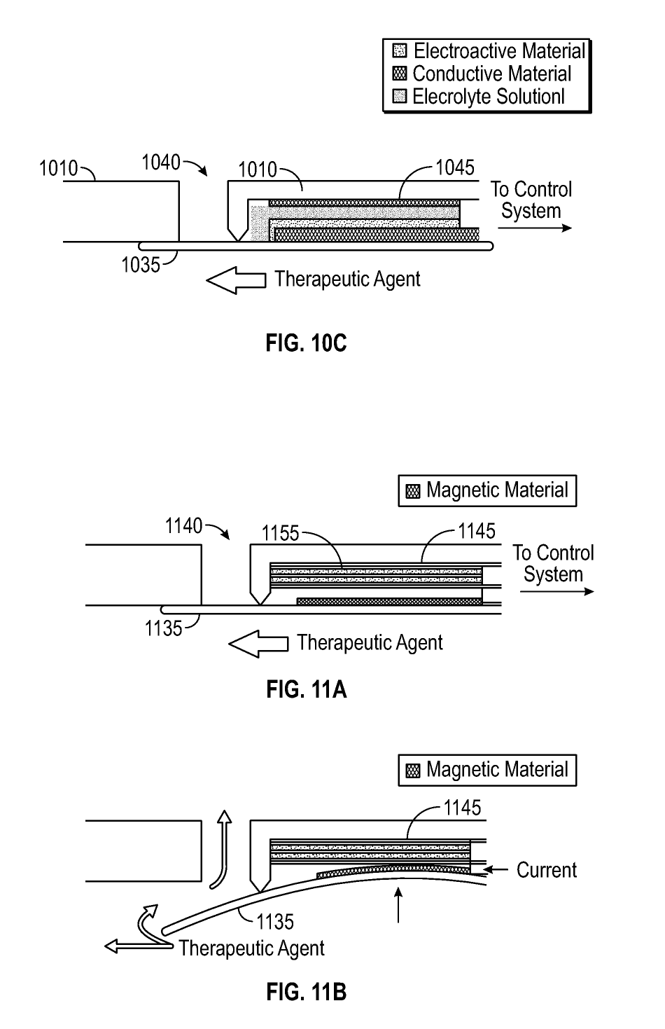

FIGS. 10A, 10B, and 10C show still another variation of a gatable microvalve. The microvalve has a valve actuator that includes an electroactive material (e.g., a polymer) that volumetrically expands or contracts when an electrical or electrochemical potential is applied to the electroactive material. In the example shown in FIG. 10A, the layer of electroactive material is coupled to the valve flap 1035 within a valve actuator cavity 1045, such that when the electrical or electrochemical potential is applied, the electroactive material expands to deflect the valve flap 1035 inwards to create an opening to the port 1040 in the tubular body 1010. Alternatively, the electroactive material may be coupled to the valve flap 1035 outside the valve actuator cavity, such that when the electrical or electrochemical potential is applied, the electroactive material contracts or reduces in volume, to deflect the valve flap 1035 inwards to create an opening to the port 1040. In other words, when a potential is applied to the electroactive material, the volumetric change in the electroactive material bends the surface of the valve flap 1035 on one side of the fulcrum and causes an opposite flexion in the valve flap 1035 on an opposite side of the fulcrum; this resultant flexion creates the opening to the port 1040 and allows fluid to pass through the port 1040. In the example shown in FIG. 10B, the electroactive material is an electroactive polymer layer deposited on a conductive material layer, which applies an electrical potential to the electroactive polymer.

In the example shown in FIG. 10C, the valve actuator cavity 1045 additionally and/or alternatively includes an electrolyte solution (e.g. sodium dodecylbenzene sulfonate, or NaDBS) that is in contact with the electroactive material and applies an electrochemical potential to the electroactive material. The valve actuator cavity 1045 is preferably a sealed, closed system that contains the electroactive material and the electrolyte solution, which reduces risk of bodily contamination in a medical application and may lead to more consistent and predictable strains during actuation. In a preferred embodiment, the electroactive material is a conjugated polymer (e.g. polypyrrole) that is doped with a mobile or immobile anion, and the conductive material may include tungsten and/or rhenium. However, any suitable thin film materials or solutions may be used. In this variation, the electroactive valve actuator has low power consumption, provides potentially relatively large amount of strain for actuation purposes, is easily scalable on the thin film level, and has a fast response time.

FIGS. 11A and 11B show still another variation of a gatable microvalve. The microvalve has a valve actuator that includes a material responsive to magnetic actuation. The valve actuator includes a thin-film solenoid 1155 that provides a magnetic force whose force may be precisely modulated by controlling current flow through the solenoid 1155. In certain examples, the valve actuator includes a layer of magnetostrictive material (e.g. ferromagnetic material) that volumetrically expands under magnetization, on a scale appropriate for the microvalve. The magnetostrictive material is coupled to the valve flap 1135 within the valve actuator cavity 1145, such that the magnetic field produced by the solenoid 1155 causes the magnetostrictive material to expand and deflect the flap inwards to create an opening to the port 1140. In certain examples, the valve actuator includes a layer of magnetic material coupled to the valve flap 1135 within the valve actuator cavity 1145 and opposite the solenoid, and/or the valve flap 1135 includes a magnetic material. The magnetic force produced by the solenoid attracts the layer of magnetic material, thereby deflecting the valve flap 1135 inwards to create an opening to the port 1140.

The strength of the magnetic field produced by a solenoid 1155 may correspond to the amount of deflection of the valve flap 1135, the resulting size of the opening, and the resulting flow rate of the fluid through the opening and out the port of the microcatheter. This second magnetic variation of the valve actuator involves consistent and predictable strains of the magnetic material in fast response to the magnetic field from the solenoid 1155, which improves effective control of the microvalve and fluid flow through the port 1140.

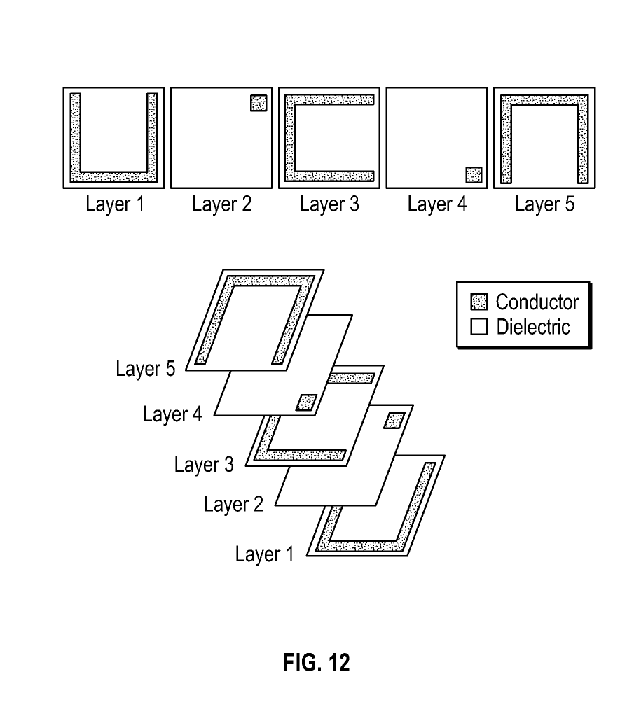

FIG. 12 shows an example of a thin film solenoid. The solenoid can be made by stacking multiple thin film sheets that include patterned conductive and dielectric material. The conductive material in successively stacked sheets preferably forms a continuous approximation of a coil shape, such as a rectangular or square coil. The example shows 5 thin film sheets to simplify the Figure. Additional thin film sheets can be used to provide additional turns of the solenoid conductor. The thin film sheets forming the solenoid are preferably deposited in the same thin film layering process during manufacture of the microcatheter, but the solenoid may alternatively be formed in a separate process and coupled to the valve actuator cavity or other suitable portion of the microcatheter.

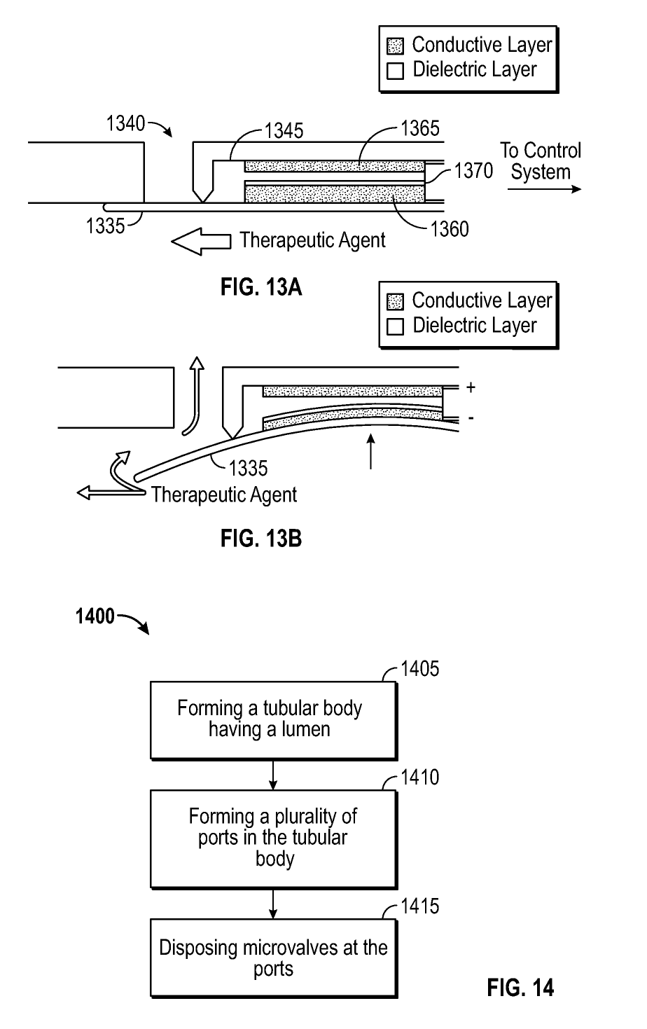

FIGS. 13A and 13B show still another variation of a gatable microvalve. The microvalve has a valve actuator that includes a material responsive to electrostatic actuation. The valve actuator may include a first conductive layer 1360 coupled to the valve flap 1335 and a second conductive layer 1365 coupled to the microcatheter wall. The first and second conductive layers are substantially parallel and separated by a small distance. The first and second conductive layers may be included in a valve actuating cavity 1345. The valve actuator may further include an insulating dielectric layer 1370 between the conductive layers to prevent shorting between the conductive layers, such as a dielectric layer deposited on one or both of the conductive layers or a third independent layer disposed between the conductive layers. The conductive layers are preferably coupled to a generator (e.g., a signal generating circuit included in a control subsystem) that selectively applies electrical potentials to the conductive layers, such that the first and second conductive layers have electrical potentials of opposite polarity. When the conductive layers are oppositely charged, the attraction between the conductive layers draws the flap of the flap valve towards the microcatheter wall, thereby creating an opening to the port 1340.

In another variation, a gatable microvalve does not include a movable valve flap. As shown in FIG. 1, the gatable microvalve can include a mesh covering a port, and the mesh can include an electroactive material. As explained previously herein, an electroactive material volumetrically expands and contracts when an electrical or electrochemical potential is applied to the electroactive material. The expanding and contracting can controllably provide and prevent fluidic transfer through mesh-covered port. For instance, the pore size of the mesh may be sized so that expansion of the electroactive material in response to an electric signal applied to the mesh causes the electroactive material to expand and close the mesh pores, thereby placing the microvalve in a closed mode. Conversely, either removing or applying a different electric potential causes the electroactive material to contract, which opens the mesh pores and places the port in an open mode. The microvalve is gatable by the opening and closing of the mesh-covered port.

FIG. 14 is a flow diagram of a method 1400 of making a device to provide targeted drug delivery, such as a microcatheter. At block 1405, a tubular body of the device is formed. The tubular body is formed having a lumen. At block 1410, a plurality of ports is formed on the tubular body, such as at a distal region of the tubular body for example. A port extends from internal to the lumen to outside the tubular body. At block 1415, a plurality of independently gatable microvalves is disposed at the plurality of ports. A microvalve can be independently controllable by providing a stimulus (e.g., an electrical or temperature stimulus) to a microvalve. The microvalve independently provides and prevents fluidic transfer through the corresponding port in response to the stimulus.

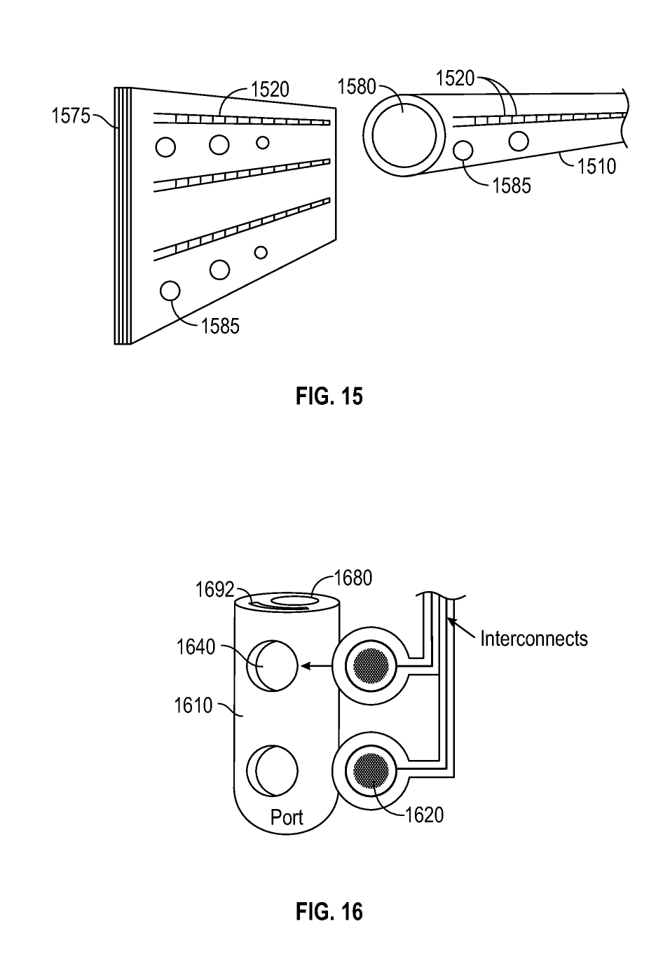

FIG. 15 shows an example of forming portions of a device to provide targeted drug delivery. In some examples, the tubular body is formed using a single sheet or substrate of thin film material. The single sheet includes one or more microvalves, the electrode sites, and the electrical conductors. The thin film sheet 1575 can be rolled and sealed to form the tubular body 1510 and define a lumen 1580 that carries fluid. The thin film sheet 1575 may be formed from biocompatible materials in a thin film layering process including deposition, patterning, etching and other techniques similar to semiconductor manufacturing processes or microelectromechanical system (MEMS) manufacturing processes. In the thin film layering process, the thin film sheet preferably defines a plurality of apertures that function as the ports and defines layers that extend over the apertures and function as valve flaps 1520 for gatable microvalves. Additional features such as electrode sites 1585 and interconnects (e.g., electrical conductors) may also be formed in the thin film layering process. Alternatively, the apertures may be formed separately and/or the valve flaps 1520 may be separate structures that are coupled to the thin film sheet, before or after the thin film sheet is rolled and sealed to form the tubular body 1510. However, the tubular body 1510, ports, and valve flaps 1520 and other features may alternatively be formed in any suitable manner. The valve flaps 1520 can be made of a somewhat flexible material such as polyimide, and are preferably on the scale of approximately 100-200 μm wide and 10 μm thick, but in alternative variations the valve flaps 1520 may be any suitable dimensions. The overall length of the tubular body may depend on the specific application of the neural drug delivery device.

Other variations of the valve actuator may include any suitable material that induce strains in response to a stimulus, and as a result induce strains in the microvalve flap. The valve actuator may be triggered by stimuli such as current, temperature, pressure, magnetic field, pH, or introduction of particular chemicals. Furthermore, although the valve actuator is preferably used in a microscale neural drug delivery device, any variations of the valve actuators may alternatively be used to as actuators in other thin-film applications. For instance, the thin-film solenoid may be utilized in other microfluidics applications. Furthermore, multiple variations of the valve actuator may be combined, such as for redundancy in control in case of failure, or increasing the maximum degree to which the microvalve is opened.

FIG. 16 shows another example of forming portions of a device to provide targeted drug delivery. The device includes a tubular body 1610 that can be formed from a flexible material such as silicone or a thermoplastic copolymer. The tubular body 1610 can be formed using one or both of extrusion and injection molding. The tubular body 1610 can be formed to have a first lumen 1682 to carry a fluid and a second lumen 1680 configured by shape and size to receive a stylet to aid in placement of the device. In certain examples, the tubular body 1610 is formed from a rigid or semi-rigid material so that a stylet is not required for placement. One or more ports 1640 can be formed in the tubular body 1610 (e.g., a sidewall of the tubular body) by laser microdrilling. In a non-limiting example, the port can be sized to have diameter in a range from 100-300 μm (e.g., 250 μm).

In an alternative method to form a microvalve, a mesh can be formed using microfabrication processes, such as material deposition, etching, etc., used in manufacturing semiconductor devices. In a non-limiting example, the mesh crossbar gap (e.g., pore size) of 1-15 μm, and a crossbar thickness of 2-20 μm. The mesh can include gold. An electroactive polymer (e.g., polypyrrole) can be deposited on the mesh to form a mesh microvalve 1620. The mesh can be roughened to better retain the electroactive polymer. The mesh can be adhered (e.g., epoxied) to the tubular body 1610 to cover a port 1640. The microvalves 1620 can be coupled to interconnects to provide independent control of the microvalves. In some variations, a single substrate is formed that contains the microvalves (e.g., movable flaps or mesh), electrode sites, and associated electrical conductors. The substrate may be a sheet of thin-film material and the components of the substrate can be formed using microfabrication techniques, such as techniques used to form semiconductor devices or MEMS for example. The tubular body can be formed to include ports that correspond to the placement of the microvalves. The single substrate can then be placed (e.g., adhered) to the tubular body.

As described previously herein in regard to FIG. 2, a neural drug delivery system 200 may include a control subsystem 230 and electrical interconnects and/or leads that carry signals between the control sub system and one or both of valve actuators and electrode sites. The control subsystem 230 can enable selective and independent control of the valve actuators, and may be one or both of programmable and manually controlled. For instance, the control subsystem 230 may modulate the amount of current provided to a conductive layer or solenoid in any particular one or more valve actuators of any variation, to transition respective microvalves between closed and open modes, and to modulate the degree to which the respective microvalves are open. In this manner, the control subsystem 230 can enable the user to control the location of open microvalves to selectively allow transfer of a fluidic therapeutic agent to target tissue, the rate at which the transfer occurs, and the duration of time over which the transfer occurs. The control subsystem 230 may further include an electrical subsystem that performs signal processing on signals such as those from the electrode sites. The interconnects and/or leads of the neural drug delivery system may be at least partially embedded in a microcatheter portion to carry signals from the control subsystem 230 to valve actuators and/or electrode sites, although at least a portion of the interconnects and/or leads may be external to the microcatheter 205 and external to the body.

The systems and devices described herein provide for highly controllable and therefore precisely targeted drug delivery. A therapeutic agent or agents can be delivered to the central nervous system with precise spatial or regional selection, and can deliver the therapeutic agent(s) at appropriate dosage levels over an appropriate amount of time.

### ADDITIONAL NOTES AND EXAMPLES

Example 1 can include subject matter (such as an apparatus or device) comprising a tubular body having a lumen and a distal region, a plurality of ports at the distal region of the tubular body, and a plurality of independently gatable microvalves disposed at the plurality of ports. A port extends from internal to the lumen to outside the tubular body, and a gatable microvalve is controllable by a stimulus to provide and prevent fluidic transfer through the ports.

In Example 2, the subject matter of Example 1 optionally includes a gatable microvalve controllable by an electrical stimulus to provide and prevent fluidic transfer through the ports.

In Example 3, the subject matter of Example 2 optionally includes a gatable microvalve that includes a movable valve flap configured to controllably provide and prevent fluidic transfer through a port. The movable valve flap includes a polymer material configured to actuate according to an electrical signal.

In Example 4, the subject matter of Example 3 optionally includes a movable valve flap that covers the port on a side of the port internal to the lumen.

In Example 5, the subject matter of Example 3 optionally includes a movable valve flap that covers the port on a side of the port external to the tubular body.

In Example 6, the subject matter of one or any combination of Examples 3-5 optionally includes a valve actuator cavity that is deformable in response to the electrical signal. The optional deforming of the valve actuator cavity changes a state of the movable valve flap from a closed mode to an open mode or from the open mode to the closed mode.

In Example 7, the subject matter of Example 6 optionally includes a valve actuator cavity that includes an electrolyte solution.

In Example 8, the subject matter of one or any combination of Examples 3-7 optionally includes a gatable microvalve that includes a shape memory alloy configured to change a state of the movable valve flap from a closed mode to an open mode or from the open mode to the closed mode according to the electrical signal.

In Example 9, the subject matter of one or any combination of Examples 3-8 optionally includes a gatable microvalve that includes a shape memory alloy and a valve actuator cavity that is deformable in response to an electrical signal applied to the shape memory alloy. The optional deforming of the valve actuator cavity changes a state of the movable valve flap from a closed mode to an open mode or from the open mode to the closed mode.

In Example 10, the subject matter of one or any combination of Examples 3-9 optionally includes a microvalve having an electroactive polymer coupled to the movable valve flap and configured for one or both of expanding and contracting according to an electrical signal. The one or both of expanding and contracting changes a state of the movable flap from a closed mode to an open mode or from the open mode to the closed mode according to the electrical signal.

In Example 11, the subject matter of one or any combination of Examples 3-10 optionally includes a tubular body that includes a thin-film polymer, a plurality of ports include apertures in the thin-film polymer, and movable valve flaps of the plurality of gatable microvalves that include one or more layers of thin-film polymer.

In Example 12, the subject matter of one or any combination of Examples 2-11 optionally includes a gatable microvalve that includes an electroactive polymer configured for one or both of expanding and contracting according to an electrical signal. The optional one or both of expanding and contracting controllably provides and prevents fluidic transfer through a port.

In Example 13, the subject matter of Example 12 optionally includes an electroactive polymer included in a mesh covering the port.

In Example 14, the subject matter of Example 1 optionally includes a gatable microvalve controllable through a temperature stimulus to provide and prevent fluidic transfer through the ports.

In Example 15, the subject matter of one or any combination of Examples 1-14 optionally includes one or more electrodes in the region of the plurality of ports.

Example 16 can include subject matter (such as a method, a means for performing acts, or a machine-readable medium including instructions that, when performed by the machine, cause the machine to perform acts), or can optionally be combined with the subject matter of one or any combination of Examples 1-15 to include such subject matter comprising forming a tubular body having a lumen, forming a plurality of ports at a distal region of the tubular body, and disposing a plurality of independently gatable microvalves at the plurality of ports. A port extends from internal to the lumen to outside the tubular body, and a gatable microvalve is controllable by a stimulus to provide and prevent fluidic transfer through the ports.

In Example 17, the subject matter of Example 16 optionally includes rolling a sheet of a thin-film polymer to form the tubular body, forming a plurality of apertures in the sheet of the thin-film polymer, and forming movable valve flaps as layers of the thin-film polymer sheet.

In Example 18, the subject matter of Example 17 optionally includes forming the movable valve flaps to be internal to the lumen.

In Example 19, the subject matter of Example 17 optionally includes forming the movable valve flaps to be external to the tubular body.

In Example 20, the subject matter of one or any combination of Examples 17-19 optionally includes depositing a shape memory alloy onto the movable valve flaps.

In Example 21, the subject matter of one or any combination of Examples 17-20 optionally includes forming one or more electrodes and electrical interconnect to the one or more electrodes in the thin-film polymer sheet.

In Example 22, the subject matter of Example 16 optionally includes forming a mesh using a microfabrication process, depositing an electro-active polymer onto the mesh, and adhering the mesh to the tubular body to cover a port.

In Example 23, the subject matter of any one or a combination of Examples 16-22 optionally includes forming the tubular body using flexible material, and forming a second lumen with the tubular body. The second lumen can be configured to receive a stylet.

In Example 24, the subject matter of one or any combination of Examples 16-23 optionally includes disposing a plurality of independently gatable microvalves that are controllable by at least one of an electrical stimulus or a temperature stimulus.

In Example 25, the subject matter of one or any combination of Examples 16, 19-21, 23, and 24 includes forming movable flaps in a single thin-film sheet onto the tubular body, and placing the single thin-film sheet onto the tubular body.

Example 26 includes subject matter (such as system), or can optionally be combined with the subject matter of one or any combination of Examples 1-7 to include such subject matter, comprising a tubular body, a plurality of ports, a plurality of independently gatable microvalves disposed at the plurality of ports, a plurality of electrical conductors, and a control subsystem. The tubular body has a lumen, a distal region, and a proximal end, and the ports are located at the distal region of the tubular body. A port extends from inside the lumen to outside the tubular body and a microvalve is electrically controllable to provide and prevent fluidic transfer through the ports. The electrical conductors are electrically coupled to the microvalves and extend to the proximal end of the tubular body. The control subsystem is electrically coupled to the electrical conductors and is configured to provide independent control of the microvalves.

In Example 27, the subject matter of Example 26 optionally includes one or more electrodes in the region of the plurality of ports, wherein the control subsystem is configured to provide electrical stimulation energy to the one or more electrodes.

In Example 28, the subject matter of one or any combination of Examples 26 and 27 optionally includes one or more electrodes in the region of the plurality of ports, wherein the control subsystem is configured to record at least one neural signal sensed using the one or more electrodes.

In Example 29, the subject matter of one or any combination of Examples 26-28 optionally includes a microvalve having a movable valve flap configured to controllably provide and prevent fluidic transfer through a port. The movable valve flap optionally includes a polymer material configured to actuate according to an electrical signal.

In Example 30, the subject matter of one or any combination of Examples 26-30 optionally includes a lumen configured to receive fluid from a reservoir. The subject matter also includes a gatable microvalve including an electroactive polymer configured for one or both of expanding and contracting according to an electrical signal. The one or both of the expanding and contracting controllably provides and prevents fluidic transfer through a port.

These non-limiting examples can be combined in any permutation or combination.

The above detailed description includes references to the accompanying drawings, which form a part of the detailed description. The drawings show, by way of illustration, specific embodiments in which the invention can be practiced. These embodiments are also referred to herein as “examples.” All publications, patents, and patent documents referred to in this document are incorporated by reference herein in their entirety, as though individually incorporated by reference. In the event of inconsistent usages between this document and those documents so incorporated by reference, the usage in the incorporated reference(s) should be considered supplementary to that of this document; for irreconcilable inconsistencies, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In the appended claims, the terms “including” and “in which” are used as the plain-English equivalents of the respective terms “comprising” and “wherein.” Also, in the following claims, the terms “including” and “comprising” are open-ended, that is, a system, device, article, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code can form portions of computer program products. Further, the code can be tangibly stored on one or more volatile or non-volatile computer-readable media during execution or at other times. These computer-readable media can include, but are not limited to, hard disks, removable magnetic disks, removable optical disks (e.g., compact disks and digital video disks), magnetic cassettes, memory cards or sticks, random access memories (RAM's), read only memories (ROM's), and the like.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. §1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description, with each claim standing on its own as a separate embodiment. The scope of the invention should be determined with reference to the appended claims, along with the full scope of equivalents to which such claims are entitled.

What is claimed is:

1. An apparatus comprising:
a tubular body having a lumen and a distal region;
a plurality of ports at the distal region of the tubular body, wherein a port extends from internal to the lumen to outside the tubular body; and
a plurality of independently gatable microvalves disposed at the plurality of ports, wherein a gatable microvalve is controllable by a stimulus to provide and prevent fluidic transfer through the ports.

2. The apparatus of claim 1, wherein the gatable microvalve is controllable by an electrical stimulus to provide and prevent fluidic transfer through the ports.

3. The apparatus of claim 2, wherein a gatable microvalve includes a movable valve flap configured to controllably provide and prevent fluidic transfer through a port, wherein the movable valve flap includes a polymer material configured to actuate according to an electrical signal.

4. The apparatus of claim 3, wherein the movable valve flap covers the port on a side of the port internal to the lumen.

5. The apparatus of claim 4, wherein the movable valve flap covers the port on a side of the port external to the tubular body.

6. The apparatus of claim 3, wherein the gatable microvalve includes a valve actuator cavity that is deformable in response to the electrical signal, wherein deforming of the valve actuator cavity changes a state of the movable valve flap from a closed mode to an open mode or from the open mode to the closed mode.

7. The apparatus of claim 6, wherein the valve actuator cavity includes an electrolyte solution.

8. The apparatus of claim 3, wherein the gatable microvalve includes a shape memory alloy configured to change a state of the movable valve flap from a closed mode to an open mode or from the open mode to the closed mode according to the electrical signal.

9. The apparatus of claim 8, wherein the gatable microvalve includes a shape memory alloy and a valve actuator cavity that is deformable in response to an electrical signal applied to the shape memory alloy, wherein deforming of the valve actuator cavity changes a state of the movable valve flap from a closed mode to an open mode or from the open mode to the closed mode.

10. The apparatus of claim 3, wherein the microvalve includes an electroactive polymer coupled to the movable valve flap and configured for one or both of expanding and contracting according to an electrical signal, wherein the one or both of expanding and contracting changes a state of the movable flap from a closed mode to an open mode or from the open mode to the closed mode according to the electrical signal.

11. The apparatus of claim 3,
wherein the tubular body includes a thin-film polymer,
wherein the plurality of ports include apertures in the thin-film polymer, and
wherein movable valve flaps of the plurality of gatable microvalves include one or more layers of thin-film polymer.

12. The apparatus of claim 2, wherein a gatable microvalve includes an electroactive polymer configured for one or both of expanding and contracting according to an electrical signal, wherein the one or both of expanding and contracting controllably provides and prevents fluidic transfer through a port.

13. The apparatus of claim 12, wherein the electroactive polymer is included in a mesh covering the port.

14. The apparatus of claim 1, wherein the gatable microvalve is controllable through a temperature stimulus to provide and prevent fluidic transfer through the ports.

15. The apparatus of claim 1, including one or more electrodes in the region of the plurality of ports.

16. A method comprising:
forming a tubular body having a lumen;
forming a plurality of ports at a distal region of the tubular body, wherein a port extends from internal to the lumen to outside the tubular body; and
disposing a plurality of independently gatable microvalves at the plurality of ports, wherein a gatable microvalve is controllable by a stimulus to provide and prevent fluidic transfer through the ports.

17. The method of claim 16,
wherein forming a tubular body includes rolling a sheet of a thin-film polymer to form the tubular body,
wherein forming a plurality of ports includes forming a plurality of apertures in the sheet of the thin-film polymer, and
wherein disposing a plurality of gatable microvalves includes forming movable valve flaps as layers of the thin-film polymer sheet.

18. The method of claim 17, including forming the movable valve flaps to be internal to the lumen.

19. The method of claim 17, including forming the movable valve flaps to be external to the tubular body.

20. The method of claim 17, including depositing a shape memory alloy onto the movable valve flaps.

21. The method of claim 17, including forming one or more electrodes and electrical interconnect to the one or more electrodes in the thin-film polymer sheet.

22. The method of claim 16, wherein disposing an independently gatable microvalve at a port includes:
forming a mesh using a microfabrication process;
depositing an electro-active polymer onto the mesh; and
adhering the mesh to the tubular body to cover a port.

23. The method of claim 16, including:
forming the tubular body using flexible material; and
forming a second lumen with the tubular body, wherein the second lumen is configured to receive a stylet.

24. The method of claim 16, wherein disposing a plurality of independently gatable microvalves includes disposing a plurality of independently gatable microvalves that are controllable by at least one of an electrical stimulus or a temperature stimulus.

25. The method of claim 16, wherein disposing a plurality of gatable microvalves includes:
forming movable flaps in a single thin-film polymer sheet to correspond to the plurality of ports; and
placing the single thin-film sheet onto the tubular body.

26. A system comprising:
a tubular body having a lumen, a distal region, and a proximal end;
a plurality of ports at the distal region of the tubular body, wherein a port extends from inside the lumen to outside the tubular body;
a plurality of independently gatable microvalves disposed at the plurality of ports, wherein a microvalve is electrically controllable to provide and prevent fluidic transfer through the ports;
a plurality of electrical conductors electrically coupled to the microvalves and extending to the proximal end of the tubular body; and
a control subsystem electrically coupled to the electrical conductors and configured to provide independent control of the microvalves.

27. The system of claim 26, including one or more electrodes in the region of the plurality of ports, wherein the control subsystem is configured to provide electrical stimulation energy to the one or more electrodes.

28. The system of claim 26, including one or more electrodes in the region of the plurality of ports, wherein the control subsystem is configured to record at least one neural signal sensed using the one or more electrodes.

29. The system of claim 26,
wherein the lumen is configured to receive fluid from a reservoir, and
wherein a microvalve includes a movable valve flap configured to controllably provide and prevent fluidic transfer through a port, wherein the movable valve flap includes a polymer material configured to actuate according to an electrical signal.

30. The system of claim 26,
wherein the lumen is configured to receive fluid from a reservoir, and
wherein a gatable microvalve includes an electroactive polymer configured for one or both of expanding and contracting according to an electrical signal, wherein the one or both of expanding and contracting controllably provides and prevents fluidic transfer through a port.
